(19) United States
(12) Patent Application Publication
(10) Pub. No.: US 2004/0161841 A1
(43) Pub. Date: Aug. 19, 2004

(54) Dna sequence comprising regulatory regions for expressing proteins

(76) Inventors: Leopold Dohner, Greifswald (DE); Dietmar Becher, Diedrichshagen (DE); Salah Salim, Kiel (DE); Rimantas Siekstele, Vilnius (LT); Kestutis Sasnauskas, Vilnius (LT)

Correspondence Address: NEEDLE & ROSENBERG, P.C., SUITE 1000, 999 PEACHTREE STREET, ATLANTA, GA, 30309-3915, US

(21) Appl. No.: 10/466,758

(22) PCT Filed: Jan. 16, 2002
(86) PCT No.: PCT/EP02/00400

(30) Foreign Application Priority Data
Jan. 17, 2001 (DE) 10101962.9

(51) Int. Cl. C12N 1/20

(52) U.S. Cl. 435/252.3

(57) ABSTRACT

Described is a DNA sequence which includes the triosephosphate isomerase gene including its regulatory sequences, a DNA sequence which is active as a promoter, expression and secretion systems containing said sequence and the use thereof and a process for producing proteins.

The invention concerns a DNA sequence which includes the triosephosphate isomerase gene including its regulatory sequences, a DNA sequence which is active as a promoter, expression and secretion systems containing said sequence, host cells transformed with said DNA, the use of the sequences for expression and secretion systems active in yeast cells and a process for producing polypeptides and RNA by means of such systems.

Various systems are available for the gene-technology production of proteins, for example recombinant microorganisms which carry expression plasmids in which the expression of foreign genes is controlled under the control of regulatory systems. Bacteria systems are frequently used as they are easy to multiply. They do however suffer from the disadvantage that they produce pyrogenic substances which have to be removed prior to use in the production of medicines or vaccines. In addition glycosylated polypeptides cannot be produced in bacteria. Therefore a series of other systems was also developed for the expression of polypeptides or proteins in eukaryontic cells. Besides cell cultures, yeasts are also considered in that respect, in particular the widespread yeast Saccharomyces cerevisiae, the genome of which is known in the meantime and which are available for some vectors and expression systems.

A further yeast genus which is considered by virtue of advantageous properties for biotechnological processes is Kluyveromyces. The species K.lactis and K.marxianus are classified as GRAS (Generally Recognised As Save) and can therefore be used with the same security as Saccharomyces. In addition K.marxianus can use a large number of carbon sources and energy sources for growth and is not highly temperature-sensitive. Kluyveromyces marxianus can grow at temperatures of up to 45° C. and therefore in that respect has advantages over temperature-sensitive Saccharomyces strains in terms of cultivation. The cells of fast-growing K.marxianus strains can divide every 35 minutes under optimum conditions. It will be noted that those good properties could not hitherto be put to optimum use as expression system with which proteins can be produced with a good level of effectiveness are scarcely available for that yeast species, and there is a lack of cloned and reliable, tested, strong-expression promoters.

In addition, in regard to the production of proteins and polypeptides by a gene-technology procedure it is desirable that the resulting products do not remain in the cell but are deposited into the ambient medium as then they can be obtained more easily.

Therefore the object of the invention was to provide promoters having improved effectiveness, which can control the expression of proteins and peptides in yeast cells. A further object of the invention was to provide vectors with which polypeptides and proteins can be expressed and the expressed products can be removed from the cell.

Those objects are attained with the DNA sequences produced in accordance with the invention, expression cassettes and vectors, plasmids and processes which are defined in the claims.

The subject-matter of the invention is therefore the provision of a new DNA sequence, new regulatory elements, a process for the removal of proteins from the cell and the provision of suitable expression cassettes, plasmids and microorganisms.

In accordance with a first aspect of the invention, there are provided a DNA with a sequence in accordance with SEQ ID No 1 or a subsequence thereof, preferably with at least 10 and preferably at least 100 nucleotides:

The DNA sequence in accordance with SEQ ID No 1 is a nucleic acid sequence which codes the regulatory regions and the open reading frame for the enzyme triosephosphate isomerase. Regulatory regions of that gene which are active as a promoter are to be found in the region of the nucleotides 1 to 1112.

The enzyme triosephosphate isomerase (hereinafter also referred to as TPI) is a glycolytic enzyme which take part in the breakdown of glucose and fructose, which takes place for the production of energy in cells, and is therefore wide-spread. Triosephosphate isomerase serves also to isomerise the dihydroxyacetone phosphate which occurs in the splitting of fructose-1, 6-biphosphate besides glycerine aldehyde-3-phosphate, to form glycerine aldehyde-3-phosphate which is then finally converted into pyruvate over a plurality of steps, with the production of energy. The enzyme is also involved in the metabolism of complex lipids. It is a homodimer whose subunits comprise approximately 250 amino acids.

The amino acid and nucleotide sequence of the enzyme triosephosphate isomerase (EC 5.3.1.1) from the yeast species Kluyveromyces marxianus var. marxianus was elucidated by the inventors and the latter is specified in SEQ ID No 1. Sequences of the enzyme TPI in other organisms are known; the sequence for TPI in S.cerevisiae was elucidated and identified by the number YDR050CCDS. It was found in a sequence comparison that the conformity of the TPI coding DNA sequences in various microorganisms, in regard to the open reading frame is high, but low in regard to the regulatory sequences.

In investigation of the expression of that enzyme in the specific organism K. marxianus it was found that the promoter controlling the expression of TPI is a very strong promoter and is efficient both in functional connection with foreign proteins and also in cells of other yeast species and strains.

A further aspect of the invention therefore concerns the provision of a new promoter which has the nucleotides 1 to 1112 of SEQ ID No 1 or parts thereof which are active as a promoter. The promoter according to the invention can be used in per se known manner in functional relationship with foreign genes.

A DNA sequence with the nucleotides 1 to 1112 of SEQ ID No 1 is also referred hereinafter as a promoter sequence.

In accordance with the invention there is provided a promoter which is active in yeasts and furnishes an expression system which is highly variable. It is suitable inter alia for Kluyveromyces and other yeast species and can be used for example for yeast species such as Saccharomyces cerevisiae and Kluyveromyces lactis. Tests which are described in the experimental part have shown that highly effective expression of foreign protein takes place under the control of the promoter according to the invention.

The new constitutive promoter provided in accordance with the invention is suitable for the expression of recombinant proteins in yeast cells. The term promoter is used here to denote a DNA sequence from which the transcription of a gene is controlled. The term “part active as a promoter” denotes a subsequence of the promoter which in connection with an open reading frame leads to the expression of the polypeptide coded by the reading frame.

It was established that the promoter which in K.marxianus regulates the expression of TPI, hereinafter also referred to as KmTPI-promoter, has seven possible transcription start locations. They are identified in the SEQ ID No 1 as transcription starts #1 to #7. It was also found that various microorganisms use various sequence units as transcription start points, for expression purposes. The efficiency of the promoter depends inter alia on which of the transcription start points are available in regard to expression and therefore the strength of the promoter can be respectively accurately adjusted for the microorganism in which expression is to take place by selection of the subsequence portions.

For example the KmTPI-promoter in Kluyveromyces marxianus has such a strong action that a subregion of the promoter DNA with only one transcription start location already results in the expression of a polypeptide. In other Kluyveromyces species such as for example K. lactis it is advantageous if at least three and preferably at least five transcription start points are contained in the sequence. Therefore for expression in Kluyveromyces species such as K. lactis the promoter used is preferably a sequence which includes at least five transcription start locations, for example a sequence at least with the nucleotides 352 to 1112 of SEQ ID No 1.

The KmTPI-promoter provided in accordance with the invention is also functional in Saccharomyces. In order to achieve satisfactory expression however it is recommended that in organisms of that genus the KmTPI-maximum promoter with the nucleotides 1 to 1112 of SEQ ID No 1 is used.

The described nucleic acid sequences with promoter activity includes such sequences which are produced by modification, substitution, deletion or insertion or combinations thereof. For that purpose the promoter sequence can also be provided with further regulatory upstream sequences with enhancer, activator and/or repressor functions. Those sequences are considered as a sequence with promoter activity, which include a part of the claimed sequence or a derivative thereof which still acts as a promoter for the expression of proteins.

Furthermore in accordance with the invention such sequences are also considered which with the claimed nucleotide sequence or the claimed parts thereof, for example the promoter sequence, involve a homology of at least 70%, preferably at least 90% and in particular at least 95%, as long as the respective sequences also have a comparable biological function. For the purposes of the present invention homology is determined in that respect in the usual manner employing the usual algorithms. Such sequences which under stringent conditions hybridise with the sequences according to the invention are also part of the invention.

In accordance with the invention moreover such nucleic acids are also considered, which code polypeptides homologous to TPI, in particular those with at least 80% homology. The expression “homologous polypeptide” in the present description includes a polypeptide which has substantially the same amino acid sequence and substantially the same biological activity as the claimed polypeptide. A homolog can be distinguished from the starting polypeptide insofar as it has more, fewer or other amino acids, but in that respect the function is retained. The man skilled in the art is aware of processes for producing suitably modified polypeptides.

The preparation of the promoter according to the invention is effected in per se known manner, insofar as either the naturally occurring sequence is isolated, which is preferred, or the sequence is produced by gene technology or chemically synthesised. Production or synthesis processes are known to the man skilled in the art and therefore do not need to be described in greater detail here. The only essential consideration is that the promoter of triosephosphate isomerase from K.marxianus, with the nucleotides up to and including 1112 of SEQ ID No 1, or an active part thereof is used.

To facilitate use of the promoter according to the invention there is further provided an expression system or an expression cassette which has the promoter sequence as defined above or a part thereof active as a promoter, a terminator and optionally further regulatory sequences such as enhancers and an insertion cloning site. It is possible to insert into the insertion cloning site the desired gene to be expressed or the DNA for a foreign protein to be expressed, which can be transcribed under the control of the promoter according to the invention.

In its simplest form the expression cassette according to the invention includes a promoter sequence or a part thereof active as a promoter, an insertion cloning site into which the polynucleotide for the protein to be expressed can be cloned, and a nucleotide sequence effective as a terminator. Sequences suitable as an insertion cloning site are known to the man skilled in the art and do not need to be described in greater detail here. For example the sequence acting as the terminator in the expression of TPI can be used as the terminator. The latter sequence includes the nucleotides 1860 to 2163 of SEQ ID No 1 or parts thereof which are active as a terminator. Good results were also achieved when using the terminator region of the endopolygalacturonase gene from Kluyveromyces marxianus.

The expression cassette according to the invention can be used in many different ways. The insertion cloning site is a section location at which the sequence can be cut open and the polynucleotide for the desired protein or peptide can be bound in. In that simplest form the protein is intracellularly produced in the expression procedure and not removed from the cell. It can then be obtained after decomposition of the cell in per se known manner. This embodiment is suitable both for small peptides and proteins which are unstable outside the cell and also for proteins which are generally intracellularly located.

The expression cassettes according to the invention can be used for the expression of DNA sequences in cells, in particular yeast cells. The expression cassettes according to the invention are particularly suitable for expression in yeast cells of the genera Saccharomyces and Kluyveromyces, for example S.cerevisiae, K.lactis, K.marxianus and others.

The expression cassette according to the invention is suitable both for incorporation in autonomously replicating plasmids and also for incorporation in yeast chromosomes by way of integrative vectors.

It is however preferable to amplify an expression cassette into which the desired polynucleotide was bound for the expression of a peptide or protein, in an E.coli plasmid and then to obtain the E.coli plasmids, to cut out the expression cassette with suitable restriction endonucleases for which section locations are provided at the edges of the expression cassette, and to bind the expression cassette into a yeast vector. The vectors usually contain selection markers in order to be able to select successfully transformed cells in per se known manner.

The plasmids can possibly multiplied in E.coli and then used in Kluyveromyces marxianus or another Kluyveromyces strain or also another yeast strain. The transformation system used for example can be known plasmids based on the Kluyveromyces drosophilarum-plasmid pKD1. Descendants of that plasmid are suitable for use in Kluyveromyces marxianus and, when the expression system according to the invention is used, lead to effective expression of foreign proteins in the corresponding host.

In another embodiment, the expression cassette including the polynucleotide to be expressed can be cut out of the plasmids according to the invention which have been prepared as described above, and brought directly into contact with yeast cells as linear or circularised DNA as an integration cassette in order to be taken up by the cells. Stable incorporation into the host cell can then be effected by way of homologous recombination if a part of the nucleic acids is homologous for the host cell. By virtue of homology of parts of the expression cassette, for example the TPI gene or the regulatory sequences thereof, with the genome of the yeast, then in a part of the treated cells the DNA is taken up into the corresponding chromosomes by interchange with the corresponding sequences.

The expression cassette according to the invention is stably incorporated into chromosomes and, when the cells are cultivated under optimum conditions, results in a good yield of the desired protein. The number of copies of the system can be set in dependence on the nature of the protein or peptide to be expressed. If a higher number is wanted, sequences of a gene which is present in a larger number of copies in the chromosome set, for example for rDNA, are ligated in per se known manner to the ends of the expression cassette in order to implement a higher number of interchange events.

In addition a marker gene can also be incorporated into the sequence in per se known manner so that the successfully transformed cells can be selected. Processes and markers which are suitable for that purpose are known to the man skilled in the art and do not need to be described in greater detail here.

The expression system according to the invention is suitable for the expression of various heterologous and homologous proteins. The expression of homologous proteins is advantageous when expression of a protein present in the organism used is to be strengthened as the promoter according to the invention can greatly improve the amount of protein produced. Preferably the system is used for the expression of hormones, for example growth hormones and growth factors, immunomodulating factors, for example interferons and interleukins, enzymes, for example endopolygalacturonase, reporter genes, for example EGFP, or antigens, for example surface antigens of viruses, inter alia S-antigens of hepatitis B virus or virus-protein 1 from polyoma virus. The latter proteins can be used to particular advantage as vaccines.

The expression cassette according to the invention is suitable inter alia for yeasts of the strains Kluyveromyces and Saccharomyces and is preferably used in yeast strains of the species Kluyveromyces marxianus var. marxianus.

TPI is an intracellularly acting enzyme and catalyses metabolic processes which normally take place within the cell. TPI is therefore usually not expected and is also not found in the medium surrounding the cell. Therefore, as is not to be expected otherwise in an enzyme with intracellular action, no signal sequence was also found.

Surprisingly now in a specific microorganism, namely Kluyveromyces marxianus, TPI was detected in the cell supernatant. Thereupon a search was made in the open reading frame which codes TPI for signal sequences, but it was not possible to find typical sequences. Therefore, removal of that enzyme which is also referred to hereinafter as KmTPI seems to be based on other Golgi-independent mechanisms. The present invention makes use of the property of the enzyme KmTPI to leave the cell.

Naturally peptides and proteins are removed from the cell when they have a signal sequence. Upon translation that signal sequence produces a segment which provides for membrane contact and passage of the protein to be removed. Evidently however that mechanism does not occur in the case of KmTPI but the specific triosephosphate isomerase from K.marxianus has properties which permit penetration of the cell membranes. In addition it was established that a peptide or protein attached by fusion is also brought out of the cell together with the triosephosphate isomerase.

The inventors of the present invention established that on the one hand KmTPI is not only removed in K.marxianus after expression from the cell but that after transformation of other yeast strains with autonomously replicating plasmids which can express the KmTPI gene, the situation involves over-expression and dumping of TPI into the culture medium, and that on the other hand it is possible by the formation of fusion proteins with TPI, to remove foreign proteins after transcription and translation as fusion proteins into the ambient cell medium.

Therefore a further subject-matter of the invention is a process for the removal of a foreign protein in the form of a fusion product with triosephosphate isomerase, in which expression is caused in respect of a sequence, the at least regulatory sequences and a fusion DNA which codes for triosephosphate isomerase and the desired peptide or protein, the fusion protein formed is isolated and the foreign protein is separated off.

It is possible in that way to obtain a fusion product in the cell supernatant. The fusion product can be both a hybrid molecule, that is to say comprise a homologous and a heterologous component, and also a fusion protein which comprises two or possibly more heterologous parts. In the fusion protein the TPI part can be present at the foreign protein both N-terminally and also C-terminally. The TPI part at the N-terminal end of the foreign protein is preferred for removal from the cell. The fusion product can then be separated in per se known manner.

In order to facilitate separation of the fusion product, a DNA sequence which codes a spacer is preferably introduced in per se known manner between the two DNA sequences coding the proteins. The spacer between the two molecule parts should be sufficiently large to permit easy separation and in a particularly preferred embodiment affords a section location for enzymes.

It was surprisingly found that by coupling to triosephosphate isomerase even hydrophobic peptides or proteins which when using ordinary signal sequences are not to be found or are to be found only to a slight degree in the supernatant are removed from cells. That was shown for the surface antigen of hepatitis B (Hbs) which cannot be removed from the cell with the usual processes.

The removal of proteins by way of fusion with TPI is therefore particularly suitable for such proteins which are normally not removed from the cell by virtue of their hydrophobicity or lacking signal peptides.

In the experimental part, expression tests are set forth, which lead to high yields when using these combinations according to the invention.

If it is desirable to remove a polypeptide or protein after expression from the cell, that is therefore implemented by producing a fusion product of KmTPI and the desired foreign protein. For that purpose, for example a polynucleotide coding a fusion protein, with N-terminal or C-terminal TPI, can be bound into the insertion cloning site of the above-described expression cassette. Preferably, a DNA sequence coding a linker is also provided between the sequences for the two proteins in order later to facilitate cleaving the fusion product. The foreign protein is then carried out of the cell after expression by virtue of the “removal capability” of KmTPI together with same as a fusion product and can be obtained in an elegant manner after separation from the cell medium and by separation of KmTPI.

It will be appreciated that a per se known signal sequence can also be used for removal of an expressed protein. It is bound in per se known manner between the promoter and the foreign gene to be expressed. Preferably the signal sequence used is one which is homologous for the organism used. Particularly good results are achieved with a combination of the promoter of the triosephosphate isomerase or a part thereof which is operative as a promoter, and the signal sequence of the endopolygalacturonase gene from Kluyveromyces marxianus.

A further improvement in expression and secretion is achieved if the removal action of TPI is reinforced by the provision of a signal sequence. In accordance with the invention therefore it is also possible to use the above-described embodiment in which a DNA sequence which codes a fusion product of KmTPI with the desired foreign protein in combination with a known signal sequence, as described in the preceding paragraph. This combination is also part of the present invention.

In a further embodiment of the invention therefore an expression and secretion system is furnished, which in operative conjunction has the above-defined promoter sequence or a part thereof active as a promoter, a sequence active as a terminator and, between those two sequences, a signal sequence. Preferably a known sequence is used as the signal sequence. This involves the signal sequence of the enzyme endopolygalacturonase (EPG) from K.marxianus. Preferably therefore the signal sequence of the enzyme EPG from K.marxianus is used.

In both the above-mentioned embodiments cultivation is effected in per se known manner, wherein either the protein is continuously delivered into the medium in a continuous process and can be continuously obtained from the fermentation liquor or in a discontinuous process the cells are cultivated, harvested and then the protein can be obtained from the liquor.

The system according to the invention is highly variable. Thus for example only the above-defined promoter sequence according to the invention or a part thereof active as a promoter can be combined together with other nucleic acid sequences which provide further regulatory sequences and a heterologous nucleotide sequence. The promoter sequence defined according to the invention or a part thereof active as a promoter can be combined with a terminator sequence, for example that defined above, in order to provide a system which is homologous for Kluyveromyces marxianus and into which the polynucleotide for the protein to be expressed is introduced, or however a system comprising the promoter according to the invention, a signal sequence and a terminator can be combined together with a gene to be expressed, which codes a desired protein, in order to produce a protein which is to be delivered into the culture. Finally the promoter sequence according to the invention or a part thereof active as a promoter can be combined with a terminator sequence and a nucleic acid of KmTPI and the desired foreign gene.

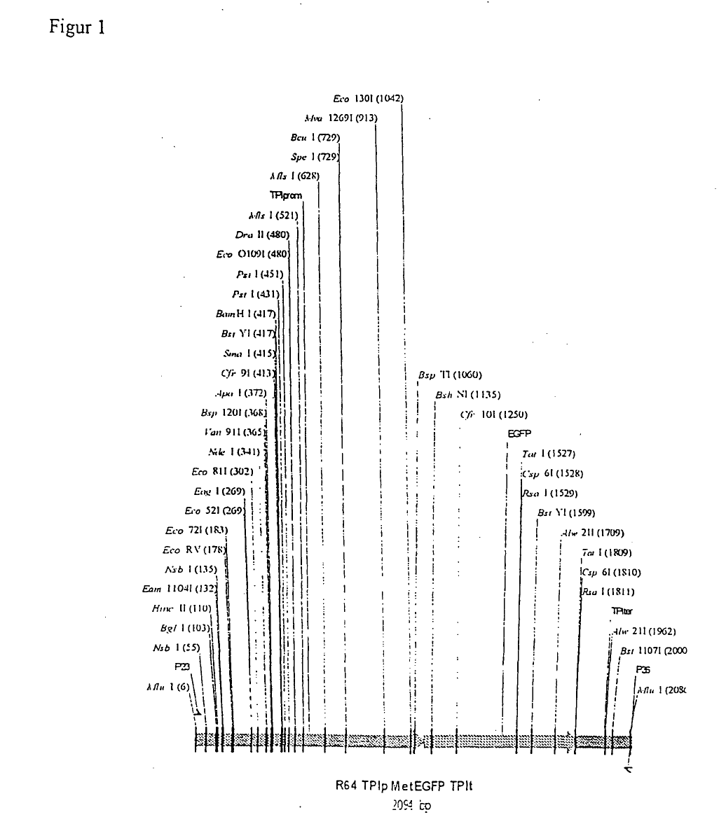

A further subject-matter of the invention is plasmids which contain expression systems according to the invention, in particular plasmids which contain the promoter, the TPI gene and the terminator of TPI from K.marxianus. Examples are the plasmids R64, R53, R48 and R11 described in greater detail with reference to FIGS. 1 to 4. Those plasmids are recombinant plasmids and can be used in the present form for amplification of the expression cassettes and for production of the proteins coded by the bound-in DNA in yeasts.

The plasmid pD1 which contains the sequence in accordance with SEQ ID No 1 was deposited in E.coli DH5a as a host cell at the Deutsche Sammlung von Mikroorganismen (DSMZ) (“German Microorganism Collection”) on Jan. 4, 2001 under deposit No DSM 13973.

As Kluyveromyces marxianus cells can grow with many different C-sources and are not highly demanding in terms of further nutrients, and in addition are temperature-insensitive, a highly effective system is afforded here. The reliable expression of the foreign proteins is achieved by the regulatory sequence of the invention, which is produced in accordance with the invention.

In accordance with the invention there is provided a system which makes it possible to use the yeast species Kluyveromyces marxianus which is highly promising as a host by virtue of its unusual physiological performances.

The system according to the invention is suitable for the expression of RNA, peptides, polypeptides, proteins and hybrid molecules including glycosylated proteins.

Set out hereinafter are some definitions for terms which are used in the description.

The term “expression vector” is used to denote a DNA molecule which can be straight or ring-shaped and contains a segment which codes a sequence for a protein or peptide of interest, which is operatively joined to regulatory sequences. Those regulatory sequences include at least promoter and terminator sequences. The expression vector can additionally contain selectable markers and further regulatory elements and must permit transmission and multiplication in host cells. Replication of the expression vectors can take place autonomously or by integration into the host genome.

The expression “DNA” or “polynucleotide” includes polymer forms of deoxyribonucleotides of any length and any modification in single- and double-strand form.

In this description “secretion vector” denotes an expression vector which in addition to the expression of a polypeptide also provides for removal of the polypeptide in the form of a foreign protein or by the signal sequence.

The expression “operatively joined” means that the individual segments are so arranged that they serve the intended purpose, that is to say they can initiate and terminate transcription and can promote expression or permit expression and secretion.

The claimed sequences can have further short sequences which do not interfere with the biological activity of the molecule. In addition the claimed sequences also include allelic variants of the sequence, that is to say alternative forms of the gene which have occurred due to mutation.

The term “protein or peptide” relates to a molecular chain of amino acids with biological activity. The expression polypeptide usually denotes amino acid sequences with up to 200 amino acids while longer chains are generally referred to as proteins. In the present description the expression polypeptide is also intended to cover proteins or conversely the expression proteins is also intended to embrace polypeptides. The proteins and/or polypeptides can be modified in vivo or in vitro, for example by glycosylation and phosphorylation.

Reference to a gene to be expressed under the control of the promoter according to the invention denotes any DNA, the expression of which is wanted. In that respect “foreign DNA” is any DNA which is normally expressed not under the control of the TPI promoter. Foreign DNA can include genes, parts of genes, fused genes, cDNA or other DNA sequences, as well as DNA which code polypeptides or proteins or also RNA or anti-sense RNA. Foreign DNA can also be a reporter gene. The foreign DNA can be a naturally occurring, gene-technically produced or chemically synthesised sequence or a combination thereof. The nucleotide sequence used and the process for the production thereof are not critical.

A further subject-matter of the invention is a process for producing a recombinant protein which is characterised in that a yeast cell is transformed with an autonomously replicating plasmid which includes an expression cassette according to the invention and a polynucleotide which codes a foreign protein, the yeast cell is cultured under conditions which are suitable for expression of the foreign protein and the protein is obtained.

Subject-matter of the invention is also a process for producing a recombinant protein which is characterised in that an expression cassette according to the invention is introduced into a yeast cell where the expression cassette is incorporated into the genome of the host cell, the cell is cultured and then the protein is obtained. Particularly preferably the expression cassette according to the invention is used as a module which permits the construction of episomal or integrative expression vectors which contain the regulatory sequences according to the invention and optionally signal sequences.

As shown in the Figures and the examples use of the promoter according to the invention in combination with a sequence for a desired protein and with a terminator leads to expression of the desired protein, in particular in yeast cells. In order to check how strong the promoter provided in accordance with the invention is the expression of the gene EGFP (enhanced green fluorescent protein) under control of the CMV (cytomegaly virus) promoter was compared to that of the EGFP gene under the control of the TPI promoter. It was found in that case that expression under the TPI promoter according to the invention produces about 50 times the expression of the CMV promoter, which shows that the promoter according to the invention leads to a boosting effect in terms of the expression of foreign proteins.

The invention is described in greater detail with reference to the following Figures and the examples.

In the Figures:

FIG. 1 is a diagrammatic view of an expression cassette (R64) according to the invention in which a functional part of the KmTPI maximum promoter (positions 21 to 1115 in accordance with SEQ ID No 1), the reading frame of the EGFP gene and a functional part of the KmTPI terminator (positions 1860 to 2127 in accordance with SEQ ID No 1) are operatively joined together.

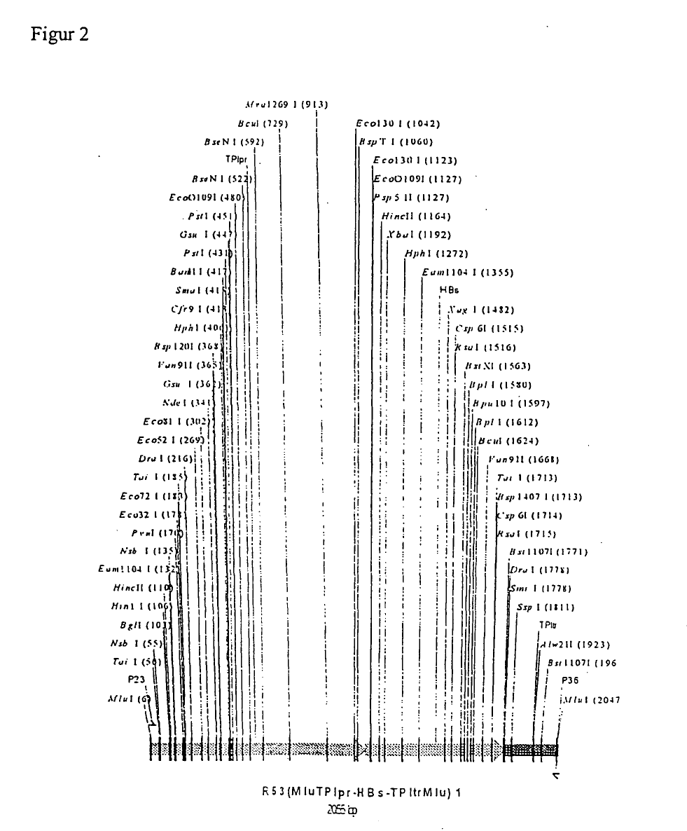

FIG. 2 shows the expression cassette R53 according to the invention in which a functional part of the KmTPI maximum promoter (positions 21 to 1115 in accordance with SEQ ID No 1), the reading frame of the hepatitis B virus S-antigen and a functional part of the KmTPI terminator (positions 1860 to 2127 in accordance with SEQ ID No 1) are operatively joined together.

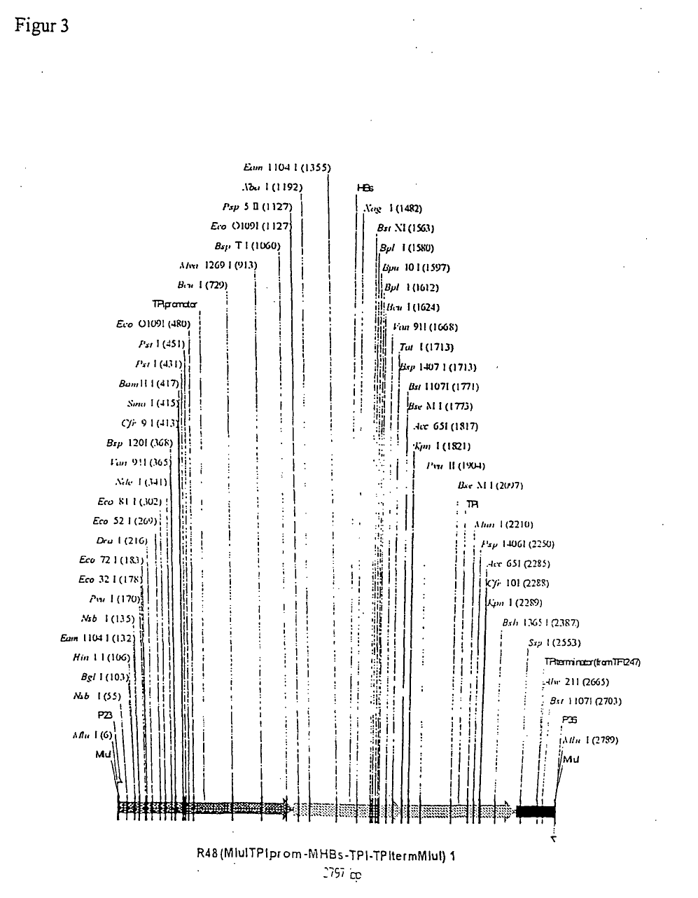

FIG. 3 shows the expression cassette R48 according to the invention in which a functional part of the KmTPI maximum promoter (positions 21 to 1115 in accordance with SEQ ID No 1), the reading frame of the hepatitis B virus S-antigen, the reading frame for triosephosphate isomerase without start methionine (positions 1116 to 1859 in accordance with SEQ ID No 1) and a functional part of the KmTPI terminator (positions 1860 to 2127 in accordance with SEQ ID No 1) are operatively joined together.

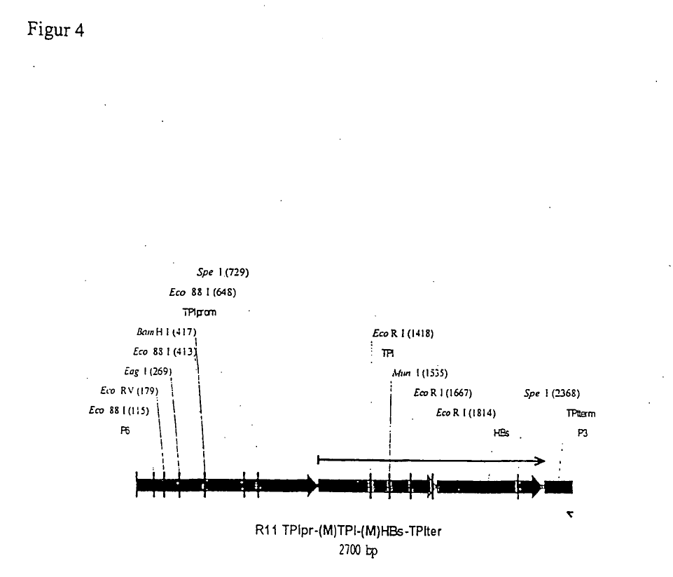

FIG. 4 shows the expression cassette R11 according to the invention in which a functional part of the KmTPI maximum promoter (positions 21 to 1112 in accordance with SEQ ID No 1), the reading frame for triosephosphate isomerase (positions 1113 to 1856 in accordance with SEQ ID No 1), the reading frame of the hepatitis B virus S-antigen, and a functional part of the KmTPI terminator (positions 1857 to 2037 in accordance with SEQ ID No 1) are operatively joined together.

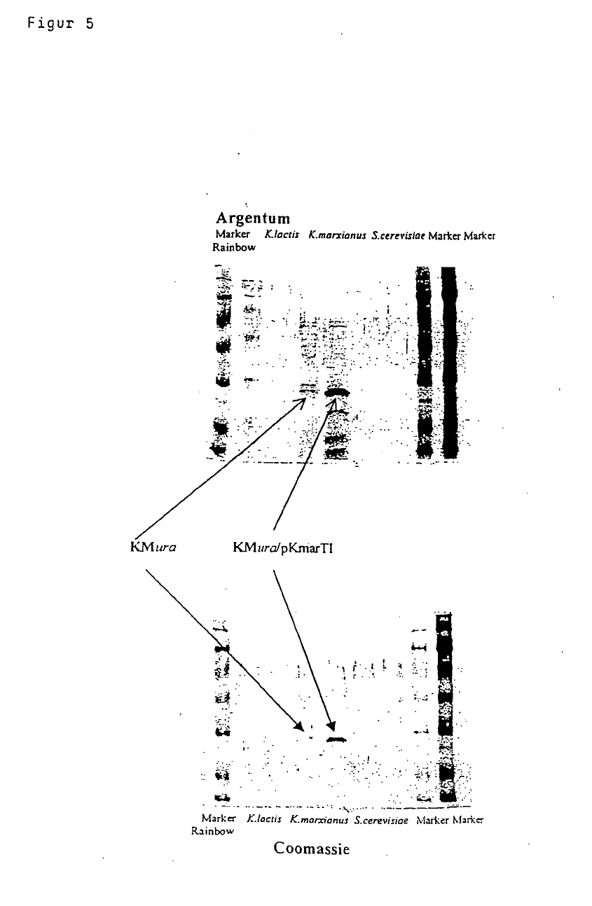

FIG. 5 shows a comparison of the expression of the TPI gene in K.marxianus initial strain and after transformation with the plasmid pKmarTI which comprises the sequence in accordance with SEQ ID No 1 and the K.marxianus vector pKDU8. Besides the culture supernatant matter of the K.marxianus strains culture supernatant matters of non-transformed strains of K.lactis and S.cerevisiae were applied in an equal amount.

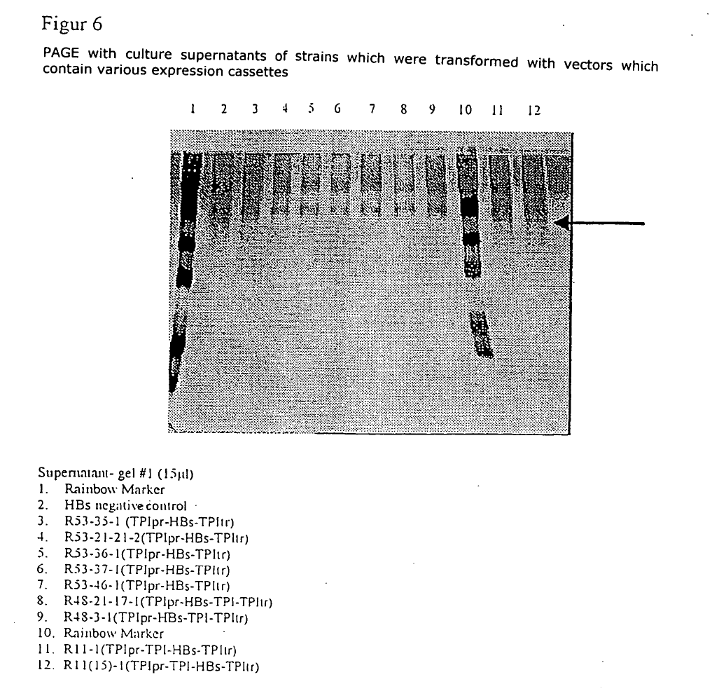

FIG. 6 shows a comparison of the secretability of HBs antigen after expression by means of various expression cassettes.

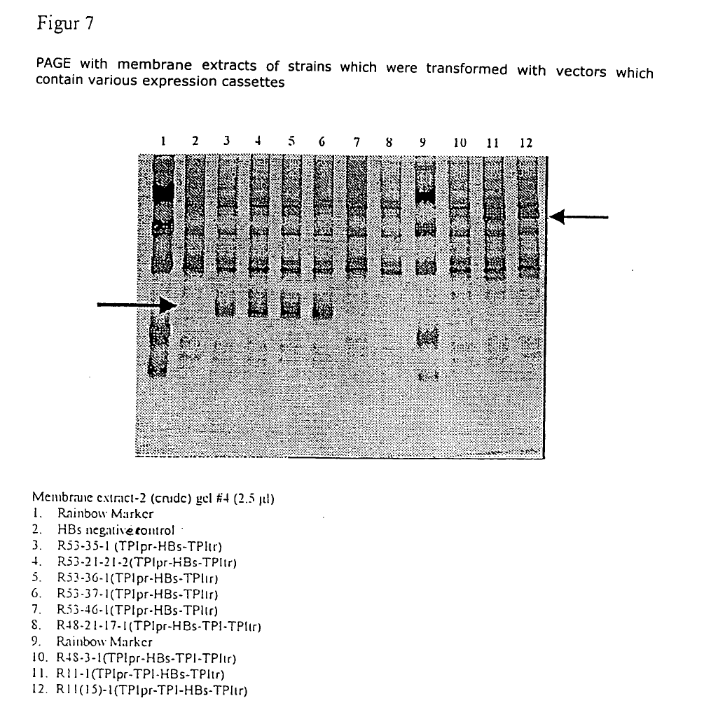

FIG. 7 shows a comparison of the expression of HBs antigen by means of various expression cassettes.

### EMBODIMENTS

### Example 1

### Construction of General Expression Cassettes

In order to provide an expression cassette with the promoter according to the invention, a DNA fragment is amplified by PCR by means of the primers P23 and P36, which fragment comprises promoter, coding region and terminator of the KmTPI gene and is delimited at the ends by artificial restriction enzyme recognition loci for MluI. That fragment is cloned into the MluI site of the plasmid pSL1180. Disposed in the coding region of the KmTPI gene is a restriction recognition locus for the restriction enzyme AclI (AACGTT). Introduced at that locus by PCR is a silent mutation which destroys the AclI site but does not alter the amino acid valine coded by GTT (MCGTT to MCGTC). A site for AclI can now be generated by PCR directly before the start codon as it is here that the nucleotide sequence MC ATG is disposed (MCgtt ATG).

An artificial AclI locus can also be generated by means of PCR immediately prior to the stop codon of the TPI coding region (GTC TAA) (aacGTt TAA). Joining the MluI/AclI promoter fragment to the AclI/MluI terminator fragment affords an empty expression cassette with a unique AclI locus for the insertion of foreign reading frames (cassette=pTIMex).

The KmTPI cassettes with unique AclI locus prior to the start codon or prior to the stop codon represent the secretion cassettes which permit an expression of foreign proteins as fusion proteins with TPI at the N- or C-terminal end and lead to a secretion of the corresponding fusion proteins (cassettes=pTIMfusN and pTIMfusC).

The cassettes can be incorporated in known manner by way of MluI into corresponding integrative or autonomously replicating vectors and can be introduced with same into recipient cells.

### Example 2

### Generation of Expression Units

The generation of expression units can be effected on the basis of the regulatory sequences of the KmTPI also in general terms by mutually overlapping PCR reactions by means of hybrid primers without AclI section locations having to be generated in the sequence. Corresponding procedures and PCR processes are known to the man skilled in the art. The system described in Example 1 is therefore not to be viewed as exclusive but only by way of example.

Example 3

### Special Expression Cassettes

Some special expression cassettes which are referred to as R64, R53, R48 and R11 were generated by PCR-procedures. Those cassettes are characterised in greater detail by diagrams and restriction maps in FIGS. 1 to 4.

Plasmid R64 (FIG. 1)

The cassette includes the KmTPI maximum promoter with the nucleotides 21 to 1115 of SEQ ID No 1, the EGFP reading frame and the KmTPI terminator with the nucleotides 1860 to 2127 in accordance with SEQ ID No 1 (TPIpr-MetEGFP-TPItr).

Plasmid R53 (FIG. 2)

The cassette includes the KmTPI maximum promoter with the nucleotides 21 to 1115 of SEQ ID No 1 (including Met), the reading frame for hepatitis B virus S-antigen and the KmTPI terminator with the nucleotides 1860 to 2127 in accordance with SEQ ID No 1 (TPIpr-HBS-TPItr).

Plasmid R48 (FIG. 3)

The cassette includes the KmTPI maximum promoter with the nucleotides 21 to 1115 of SEQ ID No 1 (including Met), the reading frame for hepatitis B virus S-antigen to the C-terminus of which is fused the reading frame for TPI without methionine and the KmTPI terminator with the nucleotides 1860 to 2127 in accordance with SEQ ID No 1 (TPIpr-HBS-TPI-TPItr).

Plasmid R11 (FIG. 4)

The cassette includes the KmTPI maximum promoter and the entire reading frame for KmTPI, at the C-terminus of which is fused, beginning with methionine, the reading frame for hepatitis B virus S-antigen. It ends with its own stop codon (UAG). The functional part which is used here of the KmTPI terminator overlaps with the stop codon of the HBs sequence (TAATTAAATTAGG) and begins with the stop codon UM at position 1857 in accordance with SEQ ID No 1. It includes only 180 pairs of bases. That cassette was cloned by way of blunt ends into the Smal locus of corresponding plasmids (TPIpr-TPI-HBS-TPItr).

1. DNA which includes the nucleotides 1 to 2163 of SEQ ID No 1 or a subsequence thereof.

2. DNA containing the nucleotides 1 to 1112 of SEQ ID No 1 or subsequences thereof which are active as a promoter.

3. A promoter sequence according to claim 2 including at least the nucleotides 352 to 1112 of SEQ ID No 1.

4. A promoter sequence according to claim 2 or a part thereof active as a promoter for use as a regulatory region for controlling the transcription of a foreign gene.

5. A promoter sequence according to one of the preceding claims or a part thereof operative as a promoter in operative conjunction with the DNA sequence for a protein to be expressed.

6. A promoter sequence according to claim 2 or a derivative thereof in operative conjunction with the signal sequence of the EPG gene from K. marxianus.

7. DNA in accordance with SEQ ID No 1 including regulatory sequences and the open reading frame of the enzyme triosephosphate isomerase from K.marxianus.

8. DNA containing the nucleotides 1860 to 2163 of SEQ ID No 1 or a part thereof active as a terminator.

9. A yeast expression cassette containing in operative conjunction the promoter sequence according to one of claims 2 to 6, an insertion cloning location and the nucleotides 1860 to 2163 of SEQ ID No 1 or a part thereof active as a terminator.

10. A yeast expression and secretion cassette containing in operative conjunction a promoter sequence according to one of claims 2 to 6 or a part thereof active as a promoter, a signal sequence, an insertion cloning location and the terminator sequence as defined in claim 8.

11. Plasmid pD1 containing the DNA sequence in accordance with SEQ ID No 1 deposited under the deposit No DSM 13973.

12. An expression vector containing in operative conjunction a promoter according to one of claims 2 to 6, a polynucleotide which codes a foreign protein, and a terminator sequence.

13. An expression vector according to claim 12 which additionally also includes a signal sequence between promoter and polynucleotide.

14. An expression vector according to claim 13 characterised in that the signal sequence of the EPG gene of K.marxianus is contained as the signal sequence.

15. An expression vector according to one of claims 12 to 14 characterised in that the polynucleotide codes a growth hormone, a growth factor, an interferon or an antigen protein or peptide.

16. An expression vector according to claim 15 characterised in that the polynucleotide codes a hepatitis B surface antigen.

17. An expression vector according to one of claims 12 to 16 characterised in that the vector is an integrative or episomal vector.

18. An expression vector according to one of claims 12 to 16 characterised in that the vector is a plasmid replicatable in yeast.

19. An expression and secretion vector including in operative conjunction a promoter sequence according to one of claims 2 to 6, a KmTPI coding DNA 3′ or 5′ adjacent to a foreign gene and a terminator sequence according to claim 8.

20. An expression and secretion vector according to claim 19 characterised in that there is also a DNA sequence which codes a spacer between KmTPI-DNA and foreign gene.

21. A host cell transformed with a plasmid according to claim 11 or an expression vector according to one of claims 12 to 20.

22. A host cell according to claim 17 characterised in that it is a cell of the species Kluyveromyces marxianus.

23. A process for producing a recombinant protein characterised in that a yeast cell is transformed with a plasmid which contains an expression vector according to one of claims 12 to 20 with a polynucleotide which codes a foreign protein or a fusion protein, the yeast cell is cultivated under conditions which are suitable for expression of the foreign or fusion protein and the protein is obtained.

24. A process for producing a recombinant protein characterised in that a yeast cell is transformed with an expression vector according to one of claims 12 to 20, the cells which have incorporated the expression vector into the genome are selected and the transformed cells are cultivated and then the protein is obtained.

25. A process for producing a recombinant protein wherein yeast cells are transformed with a secretion vector according to claim 19 or claim 20, the fusion product resulting from expression is obtained from the supernatant matter and TPI is separated from the foreign protein in per se known manner.

26. A process according to claim 25 characterised in that a promoter and a terminator of TPI are used in the secretion vector as regulatory sequences.

27. A process according to claim 26 characterised in that a sequence according to one of claims 2 to 6 is used as the promoter and a sequence according to claim 8 is used as the promoter.

28. A process for removal of a foreign protein in the form of a fusion product with triosephosphate isomerase in which a sequence, the at least regulatory sequences and a fusion DNA which codes for triosephosphate isomerase and the desired peptide or protein is expressed, the fusion protein formed is isolated and the foreign protein is separated off.

29. A process according to claim 28 characterised in that the removal action of TPI is increased by combination with a signal sequence.

30. Use of a DNA sequence with the nucleotides 1 to 1112 in accordance with SEQ ID No 1 or a part thereof as a promoter for the expression of homologous or heterologous polypeptides in yeast cells.

31. Use of a KmTPI-coding DNA sequence in accordance with SEQ ID No 1 in operative conjunction with regulatory regions and in combination with a foreign gene for the production of a fusion product of TPI and a polypeptide which is removed after expression from the cell.

32. Use of a promoter sequence according to one of claims 2 to 6 or a part thereof as a regulatory sequence for the expression of a polypeptide in combination with a signal sequence in order to express a polypeptide and remove it from the cell.


SEQUENCE LISTING (1 sequence)

SEQ ID NO: 1 - DNA, 2163 bp, Kluyveromyces marxianus
  promoter (1)..(1112)
  misc_feature (205)..(254): transcription start #1
catgaacgtgccgaacggaagcgtaatcatcatggagctggatgaaacgctcgaccgctacgtgttgcgcaccaagctccccaaatggaccacaacagggtgcgatgatgcctcggcggcgtcaacctcgggctcgaactcttcttgcgcacagaaacactgtcaatgcgagcccaccgcgatcgtctgatatcacgtgacccatgcgtcattgtatatatagcgatttaaacaaaaaaaaaagttttgaaattggaaagttgaaagttgaaaaccgacagcggccgggtaacaatgcgcggcaactctctggcctcaggttgcctcccacttcctcctcccctctctctgccatatgtcctttccgtcggccacgccctgggcccaccctccagcctccactcttggtgaacttttttttatttcccgggatccgaaactgcagcttcccaaaaaaccctgcagcacagcagcctccagcctccactaccaggccccacccagcttcccctattgcgctcccaaacaaactggccaaaaaaaaaaaaaatagcgtataaaacacagaaaaaggcgtgatctcagcttccactatcattactggatgccccgctgcctacggctacggctacacccattggccattccatccatcaagccctcgggcataccataccacacaacctgattcagttcaaccgatccacactgtagtaacacacacacacacatacactatagactagtctataccataccataccatatacacacatacatacatacaccataccatacgatgcccatattcctctctctgcgaccggaggaggcagtttcgagtttcgagttgcgagtttatatgccttgactgacttcaagcagttgttggtttgactttctgaaatttttgttgctgcactgtgcattcatgcgtttctttgattctgtgtatataagagtggacattgtaggtatatacgaatgaaatagagagttgtttgttgataggattttcgattgggttttagtatgaattggattatttcatagcccaaggacttacaaaggacttaagatacacatacacacatacacataatctatcaaacatggctagaacattctttattggtggtaacttcaagatgaacggtaccaaggcttccattaaggaaatcgttgagagattgaactctgcttcgattccatccaacgtggaagttgtgattgctcctccagctgcctacttggaccacgctgtttctttgaacaagaaggctcaagtcagtgttgctgcccaaaacgcatacttgaaggcttccggtgctttcactggtgaaaactctgtggaacaaatcaaggatgttggtgccgaatgggttatcttgggtcactccgaaagaagaacatacttccacgaaaccgatgaattcattgctgacaagaccaagtttgctttggacagcggtgtcaaggttatcctatgtatcggtgaaaccttggaagaaaagcaaaagggtatcactttggaagttgtccaaagacaattgcaagctgttttggacaaggtccaagactggaccaacgttgttgttgcttacgaaccagtctgggctattggtaccggtttggctgctacctctgacgatgctcaagccatccaccactccatcagagaattcttggccaagaagttgagcaaggacaccgctgaaaagatcagaatcctatacggtggttccgctaacggtaagaacgctgtcaccttcaaggacaaggccgacgttgacggtttcttggttggtggtgcttctttgaagccagaattcgttgacatcatcaactccagagtctaaattaaattaggttctagtcgaaatacgaaatattaaaggaaaaaaaaataataataataaataaagcctataaagctacgatgaaatagagagtgcttttgttttggaaaatttttgaaatgaatttaacggctgtatgagcacgcgcgataatgtagtgttgttactatatgatattgtatacttatatgtagcagcaagaacccgcttatcccaataacgaaataaaaacgaagaaatagcaacagttttgtttgattctcgacacaagatagatagaaaagctagctagccatagcgaaga